(19) United States
(12) Patent Application Publication
(10) Pub. No.: US 2022/0387096 A1
(43) Pub. Date: Dec. 8, 2022

(54) TELESCOPIC STRUCTURE FOR ELECTROTOME CAPABLE OF EVACUATING SMOKE

(71) Applicant: Zhejiang Shuyou Surgical Instrument Co., Ltd., Huzhou (CN)

(72) Inventors: Ying Su, Huzhou (CN); Kongpei Lu, Huzhou (CN); Zuokai Yin, Huzhou (CN); Tai Hao, Huzhou (CN)

(21) Appl. No.: 17/775,603

(22) PCT Filed: Nov. 28, 2019
(86) PCT No.: PCT/CN2019/121393

§ 371 Date: May 10, 2022

(51) Int. Cl. A61B 18/14

(52) U.S. Cl. CPC A61B 18/148; A61B 2218/008; A61B 2017/00991

(57) ABSTRACT

Embodiments provide a telescopic structure for an electrotome capable of evacuating smoke, including an electrotome pencil body. The electrotome pencil body is hollow inside and provided with an opening end at one end; the opening end of the electrotome pencil body is provided with a limit connection part. The limit connection part is sleeved with a collar; an elastic limiter is provided between the limit connection part and the collar. The telescopic structure for an electrotome capable of evacuating smoke further includes a channel that penetrates the collar and the elastic limiter in sequence and communicates with the inside of the electrotome pencil body. The telescopic structure for the electrotome capable of evacuating smoke enables the electrotome to adjust the length of the electrotome with one hand during use, which is more convenient in use and more convenient in operation.

### TECHNICAL FIELD

The present disclosure relates to the field of medical instruments, and in particular, to a telescopic structure for an electrotome capable of evacuating smoke.

### BACKGROUND

At present, electrosurgical scalpel has basically replaced mechanical scalpel, which cuts extremely quickly, and has a good hemostasis effect, and at the same time, it can automatically sterilize the wound. However, the electrosurgical scalpel tends to vaporize the body tissue during use, producing a large amount of gas, which is easily inhaled by the operators, which is harmful to health. On the other hand, the generated gas makes the surgical field unclear, and the electrodes of the electrosurgical scalpel are often blocked by the handle or the operator's hand, which affects the surgical operation, and even causes accidental injury and electrical burns or the like.

To this end, an electrotome capable of evacuating smoke is adopted nowadays. By providing a smoke tube in the handle, and then connecting an aspirator to the distal end of the smoke tube, the gas generated during the operation can be sucked away automatically or manually. At present, there are many brands of companies at home and abroad launching the electrotome pencil capable of evacuating smoke, aiming to make the electrotome pencil have the function of sucking out the harmful fumes during surgery. Among them, in order to meet the technical needs of the electrotome pencil capable of evacuating smoke, an electrotome with retractable smoking pipe capable of evacuating smoke is provided, which is clinically meaningful to meet the needs of different depths of surgical sites, and it is convenient for doctors to adjust the length of the smoking pipe at any time according to the different surgical sites during the operation, so as to ensure the smoking effect.

Patent CN201720431517.5 discloses an adjustable smokeless surgical electrotome, including a handle and an electrotome connected to the handle. A smoking pipe is provided in the handle, and a shaft body provided along its length direction is provided in the handle. The smoking pipe is fixedly connected with the shaft body through a connecting assembly, the smoking pipe can rotate around the shaft body. The smoking pipe includes a first pipe body part, a telescopic part and a second pipe body part that are integrally connected. The end of the second pipe body extends to the outside of the handle for absorbing gas, a push button and a rotating button are provided outside the handle. The push button is linked with the shaft body, configured for making the smoking pipe telescopic and elongated. The rotating button is provided in linkage with the connecting assembly, which is used to rotate the smoking pipe around the shaft body in the handle to adjust the position of the smoking pipe. The above patent relates to a length-adjustable smokeless surgical electrotome. The above-mentioned telescopic structure adopts a telescopic tube, and the length that can be stretched during use is limited, which is inconvenient during use.

### SUMMARY

According to the technical problem proposed above, a telescopic structure for an electrotome capable of evacuating smoke is provided.

The technical means that the present disclosure takes are as follows:

A telescopic structure for an electrotome capable of evacuating smoke, comprising an electrotome pencil body, wherein the electrotome pencil body is hollow inside and provided with an opening end at one end; the opening end of the electrotome pencil body is provided with a limit connection part, the limit connection part is sleeved with a collar; an elastic limiter is provided between the limit connection part and the collar; the telescopic structure for the electrotome capable of evacuating smoke further comprises a channel that penetrates the collar and the elastic limiter in sequence and communicates with the inside of the electrotome pencil body.

In an embodiment, the channel is provided with a smoking pipe.

The above structure can make the telescopic function of the electrotome pencil capable of evacuating smoke realized during use. When in use, the user moves the collar backwards to cause relative displacement between the collar and the limit connection part of the electrotome pencil body, so that the front end of the limit connection part exerts a pressure on the elastic limiter, the pressure of the limit connection part acting on the elastic limiter deforms the elastic limiter. The deformed elastic limiter makes the diameter of the channel through which the smoking pipe passes on the elastic limiter larger, so that the smoking pipe can be extended or retracted in the channel, and the length of the electrotome capable of evacuating smoke and the position of the smoking pipe can be adjusted. After determining the position of the smoking pipe, the collar is loosened by hand, since the elastic limiter starts to reset without the action of pressure, the reset of the elastic limiter makes the collar and the limit connection part return to their original relative positions. The smoking pipe can be limited by the elastic limiter due to the smaller diameter of the channel after reset, so that it will not be displaced up and down during use.

In an embodiment, the limit connection part is provided with a limit block at an end away from the electrotome pencil body.

The front end of the limit block is against the inner side of the elastic limiter, and the limit block is clamped in the collar, so that the collar can be stably sleeved on the limit connection part, and the relative positions of the collar and the limit connection part are ensured.

In an embodiment, the smoking pipe is communicated with the electrotome pencil body through the channel provided on the electrotome pencil body, the collar and the elastic limiter.

In an embodiment, the collar is provided with a protrusion at an inner side of one end adjacent to the electrotome pencil body, the protrusion is in contact with the limit block on an inside surface.

The above protrusion is provided, aiming for arranging the limit connection part in the collar to play the role of fixing and limiting.

In an embodiment, the collar is provided with a limit groove inside, for installing the limit connection part, a spacing between the front and the rear end surfaces of the limit groove is greater than a spacing between the front and the rear end surfaces of the limit block.

The setting of the spacing facilitates the installation of the limit block and the elastic limiter, and reserves a reserved space for the deformation of the elastic limiter after being squeezed.

In an embodiment, the collar is provided with a mounting groove for mounting the elastic limiter; the mounting groove is provided at 0-1 cm from the front end surface of the limit groove.

The above-mentioned structural arrangement enables the limit connection part to be displaced relative to the collar when the collar is moved backwards, so that the elastic limiter can be pushed and deformed by the limit block. The diameter of the channel for the smoking pipe to pass through becomes larger after the deformation, the smoking pipe can be retracted.

In an embodiment, the elastic limiter is annular, and the elastic limiter has a longitudinal section of a concave arc; the channel runs through a center of the elastic limiter.

In an embodiment, the collar comprises a collar body and a collar inner sleeve; the collar inner sleeve is clamped on an inner side of the collar body, and the elastic limiter is provided between a rear side surface of the collar body and a front end surface of the collar inner sleeve.

In an embodiment, the collar inner sleeve is provided with a limit part that protrudes inwardly, and is adapted with the limit connecting part.

The present disclosure has the following beneficial effects:

1. The above-mentioned telescopic structure for an electrotome capable of evacuating smoke enables the electrotome to adjust the length thereof with one hand during use, which is more convenient to use and more convenient to operate.

2. The above-mentioned telescopic structure for an electrotome capable of evacuating smoke is simple in structure and convenient in use.

3. The above-mentioned structure uses a collar structure, such that the comfort when hand-held can be increased, and the comfort of the user when in use can be increased. The user to adjust can be facilitated.

### BRIEF DESCRIPTION OF DRAWINGS

FIG. 1 is a side structure schematic diagram showing a telescopic structure for an electrotome capable of evacuating smoke according to the present disclosure;

FIG. 2 is a schematic structural diagram showing a telescopic structure for an electrotome capable of evacuating smoke according to the present disclosure;

FIG. 3 is a schematic structural diagram showing a telescopic structure for an electrotome capable of evacuating smoke according to the present disclosure;

FIG. 4 is a schematic diagram showing an elastic limiter of a telescopic structure for an electrotome capable of evacuating smoke according to the present disclosure.

In the figures:
1—electrotome pencil body, 2—limit connection part, 3—collar, 4—elastic limiter, 5—smoking pipe, 6—channel;
21—limit block, 31—protrusion, 32—limit groove, 33—mounting groove, 34—collar body, 35—collar inner sleeve, 351—limit part.

### DESCRIPTION OF EMBODIMENTS

The following are specific embodiments of the present disclosure and the accompanying drawings to further describe the technical solutions of the present disclosure, but the present disclosure is not limited to these embodiments.

### Example 1

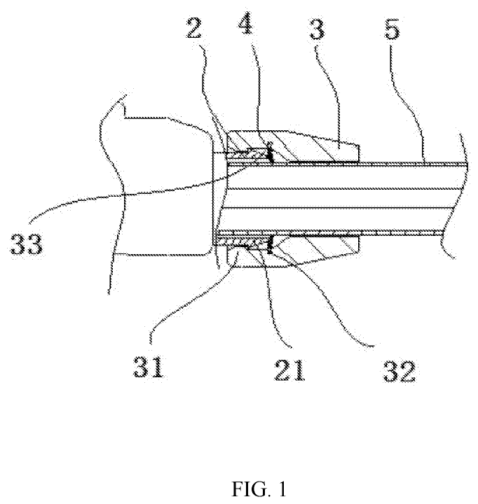

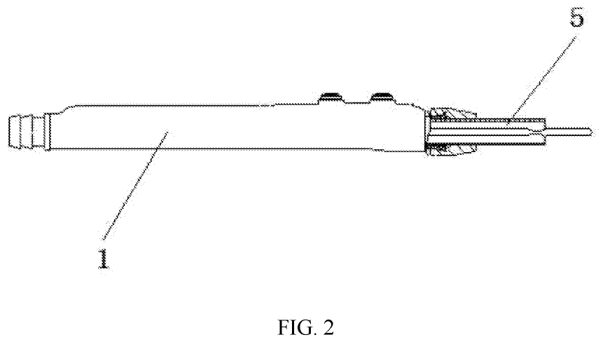

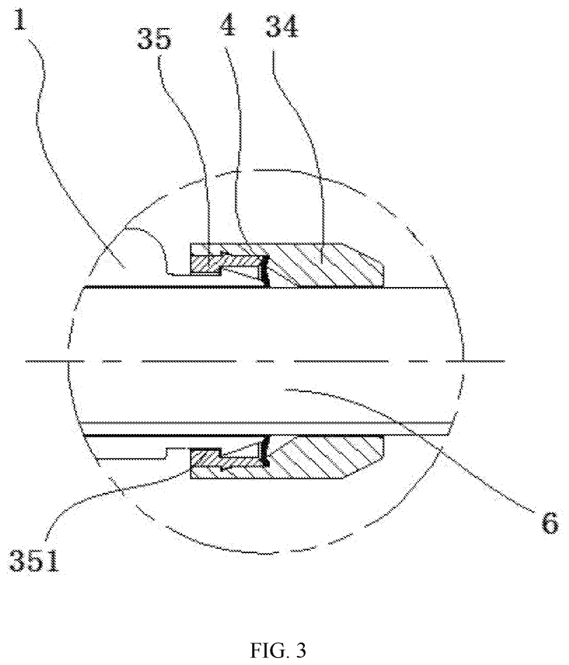

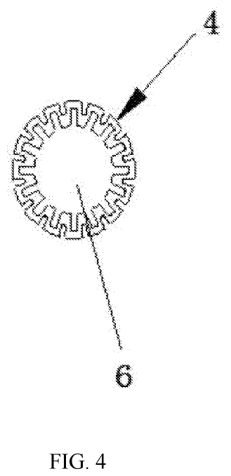

As shown in FIGS. 1 to 4, a telescopic structure for an electrotome capable of evacuating smoke may include an electrotome pencil body 1, the interior of the electrotome pencil body 1 may be hollow and one end may be provided with an opening. The opening end of the electrotome pencil body 1 may be provided with a limit connection part 2. A collar 3 may be sleeved outside the limit connection part 2, and an elastic limiter 4 may be provided between the limit connection part 2 and the collar 3. The telescopic structure for an electrotome capable of evacuating smoke may also include a channel 6 which runs through the collar 3 and the elastic limiter 4 in sequence and communicates with the inside of the electrotome pencil body 1.

A limit block 21 may be provided at the end of the limit connection part 2 away from the electrotome pencil body 1.

The limit block 21 may be in in contact with the elastic limiter 4 at one end away from the electrotome pencil body 1.

The channel provided at the electrotome pencil body 1, the collar 3 and the elastic limiter 4 may be provided with a smoking pipe 5.

The above-mentioned smoking pipe realizes retractility and positioning through the change of the aperture size of the channel on the elastic limiter by the user during use.

A protrusion 31 may be provided on the inner side of the rear end of the collar 3, and the inner side surface of the protrusion 31 may be in contact with the rear end surface of the limit block 21.

The collar 3 may be provided with a limit groove 32 for installing the limit connection part 2, and the spacing between the front end surface and the rear end surface of the limit groove 32 may be larger than that between the front end surface and the rear end surface of the limit block 21.

The collar 3 may be provided with a mounting groove 33 for mounting the elastic limiter 4. The mounting groove 33 may be 0-1 cm, preferably 0.1 cm, away from the front end surface of the limit groove 32. In another embodiment, the mounting groove 33 may be 0.2 cm away from the front end surface of the limit groove 32. In yet another embodiment, the mounting groove 33 may be 0.3 cm away from the front end surface of the limit groove 32.

The elastic limiter 4 can be annular, the longitudinal section of the elastic limiter 4 can be concave arc, and the channel through which the smoking pipe 5 can pass may be located at the center of the elastic limiter 4.

The collar 3 may include a collar body 34 and a collar inner sleeve 35. The collar inner sleeve 35 may be clamped on the inner side of the collar body 34. Between the rear side surface of the collar body 34 and the front end surface of the collar inner sleeve 35, an elastic limiter 4 may be provided.

The collar inner sleeve 35 may be provided with a limit part 351 which is protruded inward and is adapted to be matched with the limit connection part 2.

The specific embodiments described herein are merely illustrative of the spirit of the present disclosure. Those skilled in the art to which the present disclosure pertains can make various modifications or additions to the described specific embodiments or substitute in similar manners, but will not deviate from the spirit of the present disclosure or go beyond the definitions of the appended claims.

1. A telescopic structure for an electrotome capable of evacuating smoke, comprising an electrotome pencil body, wherein the electrotome pencil body is hollow inside and provided with an opening end at one end; the opening end of the electrotome pencil body is provided with a limit connection part, the limit connection part is sleeved with a collar; an elastic limiter is provided between the limit connection part and the collar; the telescopic structure for the electrotome capable of evacuating smoke further comprises a channel that penetrates the collar and the elastic limiter in sequence and communicates with the inside of the electrotome pencil body;
wherein the limit connection part is provided with a limit block at an end away from the electrotome pencil body; and
wherein the collar is provided with a protrusion at an inner side of one end adjacent to the electrotome pencil body, the protrusion is in contact with the limit block on an inside surface.

2. (canceled)

3. The telescopic structure for an electrotome capable of evacuating smoke according to claim 1, wherein the limit block is in contact with the elastic limiter at an end away from the electrotome pencil body.

4. (canceled)

5. The telescopic structure for an electrotome capable of evacuating smoke according to claim 1, wherein the collar is provided with a limit groove inside, for installing the limit connection part, a spacing between two end surfaces of the limit groove is greater than a spacing between two end surfaces of the limit block.

6. The telescopic structure for an electrotome capable of evacuating smoke according to claim 5, wherein the collar is provided with a mounting groove for mounting the elastic limiter; the mounting groove is provided at 0-1 cm from a front end surface of the limit groove.

7. The telescopic structure for an electrotome capable of evacuating smoke according to claim 1, wherein the elastic limiter is annular, and the elastic limiter has a longitudinal section of a concave arc; the channel runs through a center of the elastic limiter.

8. The telescopic structure for an electrotome capable of evacuating smoke according to claim 1, wherein the collar comprises a collar body and a collar inner sleeve; the collar inner sleeve is clamped on an inner side of the collar body, and the elastic limiter is provided between a rear side surface of the collar body and a front end surface of the collar inner sleeve.

9. The telescopic structure for an electrotome capable of evacuating smoke according to claim 8, wherein the collar inner sleeve is provided with a limit part that protrudes inwardly, and is adapted with the limit connecting part.
